(19) United States
(12) Patent Application Publication
(10) Pub. No.: US 2015/0140616 A1
(43) Pub. Date: May 21, 2015

(54) PROCESS FOR MAKING FURFURAL

(71) Applicant: ARCHER DANIELS MIDLAND COMPANY, Decatur, IL (US)

(72) Inventors: Tom Binder, Decatur, IL (US); Ahmad Hilaly, Forsyth, IL (US); William Christopher Hoffman, Decatur, IL (US); Alexandra Sanborn, Lincoln, IL (US)

(73) Assignee: ARCHER DANIELS MIDLAND COMPANY

(21) Appl. No.: 14/607,527

(22) Filed: Jan. 28, 2015

Related U.S. Application Data
(63) Continuation of application No. 14/279,559, filed on May 16, 2014.
Application No. 14/279,559 is a continuation of application No. 14/342,634, filed on Mar. 4, 2014.
Application No. 14/342,634 is a continuation of application No. 14/279,550, filed on May 16, 2014.
Application No. 14/279,550 is a continuation of application No. 14/342,634, filed on Mar. 4, 2014.
Application No. 14/342,634 is a continuation of application No. PCT/US14/48783, filed on Jul. 30, 2014.
Application No. PCT/US14/48783 is a continuation of application No. 13/521,462, filed on Jul. 11, 2012, now abandoned.
(60) Provisional application No. 61/864,228, filed on Aug. 9, 2013.
(60) Provisional application No. 61/300,853, filed on Feb. 3, 2010.

(51) Int. Cl. C12P 17/04; C07D 307/08; C07D 307/36; C07D 307/50; C12P 7/06; C12P 7/10

(52) U.S. Cl. CPC C12P 17/04; C12P 7/06; C12P 7/10; C07D 307/36; C07D 307/50; C07D 307/08; C12P 2203/00
USPC 435/126; 549/505; 549/489; 549/509

(57) ABSTRACT

Processes are described for producing furfural from a mixture of pentoses and hexoses, by dehydrating and cyclizing pentoses to provide furfural using a water-soluble acid at elevated temperatures in the presence of a low-boiling, water-immiscible organic solvent, such as toluene, which is effective for extracting the furfural into an organic phase portion. In certain embodiments, a fermentation step occurs prior to the dehydration step to convert hexoses in the mixed pentoses and hexoses to ethanol while conserving pentoses therein for making furfural.

### CROSS REFERENCE TO RELATED APPLICATIONS

In certain embodiments, the present invention is a continuing application from copending U.S. patent applications Ser. Nos. 14/279,559, 14/342,634 and 14/279,550 all to Bao et al., now published as US 2014/322766 (“US'766”), US 2014/0227742 (“US'742”) and US 2014/0322763 (“US'763”), respectively, all of which published applications are now incorporated by reference herein.

In other embodiments, the present invention is also a continuing application from copending Patent Cooperation Treaty Application Serial No. PCT/US2014/048783, filed Jul. 31, 2014 for “Process for Producing Furan from Furfural from Biomass” (hereafter the “WO'783 application”), from U.S. Provisional Patent Application Ser. No. 61/864,228, filed Aug. 9, 2013, the WO'783 application now being incorporated by reference herein.

In other embodiments, the present invention is also a continuing application from copending U.S. patent application Ser. No. 13/521,462, filed Jul. 11, 2012, now published as of May 23, 2013 as US 2013/0130331 to Binder et al., “Method of Producing Sugars Using a Combination of Acids to Selectively Hydrolyze Hemicellulosic and Cellulosic Materials” (hereafter “US'331”) which claims priority from U.S. Provisional Patent Application Ser. No. 61/300,853, filed Feb. 3, 2010, the US'331 published application now being incorporated by reference herein.

### BACKGROUND

The use of biomass—of materials whose carbon content is of biological rather than fossil origin—for providing chemicals and fuel products presently derived from fossil-origin materials such as petroleum, or for providing acceptable biobased, functional alternatives to such chemicals and fuel products, has increasingly become a focus of research and development investment and effort in recent years as supplies of fossil-origin materials have been compromised or been more difficult or expensive to acquire and use.

Certain chemical and fuel product replacements or alternatives are already produced on a large, commodity scale from biomasses. For the liquid fuel products area, for instance, ethanol and biodiesel (fatty acid alkyl esters) have heretofore been produced on a commodity scale from corn or other grains and from sugar cane (for ethanol) and from various vegetable oils and fats (for biodiesel).

It has been long recognized, though, that it would be preferable to be able to make suitable liquid fuels and fuel additives from lignocellulosic biomasses containing typically 6 percent or more of acid detergent insoluble lignin (on a dry weight basis) and which are not used as food products, or which can be harvested or sourced and used without materially adversely affecting land use patterns and behaviors (for example, deforestation to produce additional soy, corn or like crops). A number of non-food, lignocellulosic biomasses might be contemplated of this character, including, for example, purpose-grown non-food biomass crops (such as grasses, sweet sorghum, fast growing trees), or more particularly wood wastes (such as prunings, wood chips, sawdust) and green wastes (for instance leaves, grass clippings, vegetable and fruit wastes). It has been estimated in addition as to lands already under cultivation for food crops or other purposes that about three quarters of the biomass generated is waste, so that whether the biomass in question is waste in the production of a food crop or some other crop to which land has been devoted in cultivation or arises from sources unconnected to any cultivated crop, it would seem with the abundance of lignocellulosic feeds available that the various chemical and fuel products we require that could be made starting with lignocellulosic biomasses, should in fact be capable of being made economically.

As a practical matter, however, the production of the various chemical and fuel products of interest from a lignocellulosic biomass poses a number of significant challenges. A first difficulty arises from the very different characteristics of the various components comprising lignocellulosic biomasses.

In this regard, as is true of fossil-based materials such as petroleum, the practical, real-world capability of producing the full range of commodity chemicals and fuel product replacements or alternatives that are or will be needed, on the scale and with the qualities, economy and efficiency that are needed, depends to an extent on how effectively and efficiently the feedstock—lignocellulosic biomass—can be fractionated into its component parts and on how effectively and efficiently these component parts can in turn be further processed to yield the desired commodity chemicals and fuel product replacements or alternatives.

Lignocellulosic biomasses are comprised mainly of cellulose, hemicellulose and lignin fractions, with cellulose being the largest of these three components. Cellulose derives from the structural tissue of plants, and consists of long chains of beta glucosidic residues linked through the 1,4 positions. These linkages cause the cellulose to have a high crystallinity and thus a low accessibility to the enzymes or acid catalysts which have been suggested for hydrolyzing the cellulose to C₆ sugars (or hexoses) for further processing. Hemicellulose by contrast is an amorphous heteropolymer which is easily hydrolyzed, while lignin, an aromatic three-dimensional polymer, is interspersed among the cellulose and hemicellulose within a plant fiber cell and has presented the most significant challenges for further processing and upgrading.

Because of the differences in the cellulosic, hemicellulosic and lignin fractions of biomass, as well as considering other lesser fractions present in various biomasses to different degrees, as related in U.S. Pat. No. 5,562,777 to Farone et al., “Method of Producing Sugars Using Strong Acid Hydrolysis of Cellulosic and Hemicellulosic Materials”, a number of processes have been developed or proposed over the years to fractionate lignocellulosic biomasses and hydrolyze the cellulosic and hemicellulosic fractions.

Fundamentally both biological and non-biological processes have been disclosed, with the oldest and best known non-biological methods of producing sugars from cellulose involving acid hydrolysis, most commonly sulfuric acid-based hydrolysis using a dilute acid approach, a concentrated acid approach or a combination of the two. The '777 patent to Farone et al. describes the advantages and disadvantages of the various sulfuric acid-based processes then known to the art, and suggests a further variation using strong acid/sulfuric acid hydrolysis and employing one or more iterations of a combination of a decrystallization step wherein the biomass (and/or including the solids left from the decrystallization step in a previous iteration) is mixed with a 25-90 percent sulfuric acid solution to solubilize a portion of the biomass, then the acid is diluted to between 20 and 30 percent and the mixture heated to preferably between 80 and 100 degrees Celsius for a time to solubilize the cellulosic fraction and any hemicellulosic material that had not been hydrolyzed.

More recently, in several applications that are commonly assigned with the present application, we have described alternative methods for fractionating a lignocellulosic biomass and then further processing one or more of the cellulosic, hemicellulosic and lignin fractions to produce various products of commercial interest.

For example, in the published US'331 application, a method is described wherein a first, weak organic acid is applied to a lignocellulosic biomass, preferably near a collection point for the biomass, under conditions sufficient to depolymerize hemicellulosic materials and solubilize lignins in the biomass. The “cooked” acidified biomass is then dried to remove water therefrom to an extent whereby the dried solids can be pelletized for shipment to a central facility. Then, at the central facility, pelletized, weak acid-processed biomass is washed with a solvent or solvent mixture which is effective for separating the solubilized and depolymerized hemicelluloses and lignins from a cellulosic fraction of the biomass, and then the cellulosic fraction is contacted with a second, strong mineral acid (or acids) under conditions suited to providing a hexose product or stream. Preferably, the first, weak organic acid is applied to the biomass in a vapor form at elevated temperatures, in part to reduce the drying load prior to the pelletization step.

In the US'742, US'763 and US'766 published applications, a method and an improvement to that method are described for processing a lignocellulosic biomass to form an acylated cellulose pulp, that includes contacting a lignocellulosic biomass with a first amount of a C₁-C₂ acid selected from the group consisting of acetic acid, formic acid and mixtures of the same. The contacted lignocellulosic biomass is heated to a temperature and for a time sufficient to hydrolytically release a first portion of hemicellulose and lignin, forming a hydrolysate liquid and an acylated lignocellulosic cake. The acylated lignocellulosic cake is separated from the first hydrolysate liquid and is contacted with a second amount of the C₁-C₂ acid to wash hemicellulose and lignin from the acylated lignocellulosic cake. The acid wash liquid including soluble hemicellulose and lignin is separated from the acid washed cake and the cake is contacted with a first amount of a C₁-C₂ acid-miscible organic solvent to further wash the C₁-C₂ acid, hemicellulose and lignin from the acid washed acylated cake leaving an acylated cellulose pulp, which is separated from the C₁-C₂ acid-miscible solvent wash liquid. In a further embodiment, the solvent wash liquid can be combined with at least one of the hydrolysate and the acid wash liquid forming an acidic organic solvent extract. The acidic organic solvent extract is condensed, forming an acidic organic solvent syrup enriched with hemicellulose and lignin. To that syrup, a second amount of the C₁-C₂ acid-miscible organic solvent may be added, the second amount being sufficient to form a precipitate comprised of hemicellulose and lignin. The precipitate of hemicellulose and lignin is then separated from the acidic organic solvent syrup. The precipitate is mixed with an aqueous solvent to form a solution of solubilized hemicellulose and insoluble lignin and the insoluble lignin is separated from the solubilized hemicellulose.

A “C₁-C₂ acid-miscible organic solvent” referenced above is defined as a non-acidic organic solvent that is miscible with acetic acid and able to form a precipitate of hemicellulose and lignin from an acetic acid solution containing the same, with the proviso only that the C₁-C₂ acid-miscible organic solvent is not a halogenated solvent. The organic solvent used has following characteristics: the solubility of sugars in the solvent must be low, and at least a subfraction of the lignin must be partially soluble in the solvent. Such solvents are slightly polar. Preferably the solubility of water in the organic solvent should be low. Further, the polarity of the solvent should not be too low to effectively extract acetic acid from water. Suitable examples include low molecular weight alcohols, ketones and esters, such as C₁-C₄ alcohols, acetone, ethyl acetate, methyl acetate, and methyl ethyl ketone, and tetrahydrofuran.

In an improvement to the above-described method, liquid/liquid separation methods are used instead of filtration in certain steps, so that viscosity limitations inherent to the filtration process are avoided. In this regard, the solids in the concentrated hemicellulose and lignin syrup are limited to not more than about 40% by filtration-related viscosity concerns, as the C₁-C₂ acid-miscible organic solvent is removed by evaporation. By using liquid/liquid separation methods, the evaporation can be carried out until at least a concentration of 52% solids in the concentrated hemicellulose and lignin syrup is reached. The higher level of solids concentration in turn permits smaller amounts of acid and solvent to be used in subsequent purification steps. As well, substantially reduced quantities of water are needed for the water washing steps, so that the costs of recovery of acid and solvent, especially the separation of water and acid mixtures, are reduced. Still other refinements and improvements are described in addition.

### SUMMARY OF THE INVENTION

The following presents a simplified summary of the invention in order to provide a basic understanding of some of its aspects. This summary is not an extensive overview of the invention and is intended neither to identify key or critical elements of the invention nor to delineate its scope. The sole purpose of this summary is to present some concepts of the invention in a simplified form as a prelude to the more detailed description that is presented later.

As mentioned, whatever the method used for the fractionation of a lignocellulosic biomass, the common objective of all such methods is to provide a plurality of renewable feeds in sufficient purity to be further upgraded in an economic manner to chemicals and fuel products that are presently derived from fossil-origin materials such as petroleum, or to acceptable biobased, functional alternatives to these chemicals and fuel products.

Commonly, sugar syrups enriched in the pentose and hexose sugars are sought, as in the US'742, US'763 and US'766 applications, for example. The hexose sugars are readily fermented to ethanol, while the same cannot be said of the pentose sugars. Consequently, one approach taken by others in the past has been to undertake improvements in the fermentation of mixed C₅/C₆ sugars streams, recognizing that even according to the most effective methods of hydrolytically fractionating a lignocellulosic biomass, some content of pentoses will be present in any hexoses product/feed and some content of hexoses will be present in any pentoses product/feed.

With this in mind, the present invention relates in one aspect to a process for dealing with mixed C₅/C₆ sugars from a lignocellulosic biomass in a fundamentally different manner, without the need for departing from long-practiced fermentation methods for producing ethanol. Fundamentally, rather than trying to fully accommodate the presence of pentoses in the context of a fermentation of a hexose-containing feed to produce ethanol, a conventional fermentation of the hexoses in a mixed C₅/C₆ sugars feed is initially undertaken with the objective of minimal conversion of the pentoses to sugar alcohols.

In one embodiment according to this first aspect, the hexoses in the mixed C₅/C₆ sugars feed are supplemented with liquefied starch prior to the hexose fermentation step, to provide improved energy utilization in a subsequent distillation step wherein a commercial grade ethanol product is separated from the pentoses in the mixed C₅/C₆ sugars feed and from any unconverted hexoses therein. Preferably, again, minimal conversion of the pentoses present in the mixed C₅/C₆ sugars feed is sought in the fermentation step so that these are carried forward for further processing after the distillation. The remainder of the mixed C₅/C₆ sugars feed then undergoes an acid-catalyzed dehydration and cyclization to produce furfural from the pentoses. In certain embodiments, the dehydration and cyclization are accomplished with using a water-soluble acid in the presence of a low-boiling, substantially water-immiscible organic solvent. The furfural is extracted into an organic solvent phase comprising the low-boiling, substantially water-immiscible organic solvent, with recovery of unconverted hexose sugars and/or of valuable hexose dehydration products (for example, 5-(hydroxymethyl)furfural (or HMF) and levulinic acid) in an aqueous phase. High starting dry solids concentrations are achievable in certain embodiments, with nearly quantitative yields to furfural from the pentoses in a mixed C₅/C₆ sugars feed and with high accountability of the combined sugars in a biomass.

In certain embodiments, the mixed C₅/C₆ sugars feed that is fermented derives from an upstream biomass fractionation process wherein a cellulosic component of the biomass is hydrolyzed to hexoses and a hemicellulosic component of the biomass is hydrolyzed to pentoses. In particular embodiments, an upstream biomass fractionation process according to any of the US'742, US'763, US'766 or US'331 applications is used to generate the mixed C₅/C₆ sugars stream. In other embodiments, the mixed C₅/C₆ sugars feed that is fermented is not derived from a prior biomass fractionationation process, but is the direct hydrolyzate of a whole biomass.

In an alternative embodiment, the ethanol from the hexose fermentation step is combined with ethanol from a separate starch fermentation, to provide the improved energy utilization in a subsequent distillation step wherein a commercial grade ethanol product is separated from the pentoses in the mixed C₅/C₆ sugars feed and from any unconverted hexoses therein.

In a further alternative embodiment according to this first aspect, the hexoses in the mixed C₅/C₆ sugars feed are not supplemented with liquefied starch prior to the hexose fermentation step, and the ethanol from the hexose fermentation step is not recovered in a subsequent distillation step but is instead used to modify the properties of a low boiling, substantially water-immiscible organic solvent used in certain embodiments for the acid-catalyzed dehydration step and to improve the recovery of valuable hexose dehydration products in the aqueous phase, namely, 5-hydroxymethylfurfural (HMF) and levulinic acid.

In another aspect, the present invention relates to a method for making furfural from a mixed C₅/C₆ sugars feed from a lignocellulosic biomass in the absence of a preceding hexose fermentation step. In this alternate aspect, the mixed C₅/C₆ sugars feed undergoes an acid-catalyzed dehydration using a water-soluble acid in the presence of a low-boiling, substantially water-immiscible organic solvent to convert the pentoses therein to furfural. The furfural is extracted into an organic solvent phase, with recovery of valuable hexose dehydration products (for example, 5-(hydroxymethyl)furfural (or HMF) and levulinic acid) in an aqueous phase, together with any unconverted hexoses. In particular embodiments, an upstream biomass fractionation process according to any of the US'742, US'763, US'766 or US'331 applications is used to generate the mixed C₅/C₆ sugars stream. In other embodiments, the mixed C₅/C₆ sugars feed is not derived from a prior biomass fractionationation process, but is the direct hydrolyzate of a whole biomass.

In certain embodiments according to either aspect, wherein a hexose fermentation step is used or not used, the acid-catalyzed dehydration is accomplished in a plurality of reactors in series with an addition of a low-boiling, substantially water-immiscible organic solvent upstream of each reactor in the series. In a further refinement designed to reduce the energy requirements for recovering the solvent from the furfural dehydration product, after separating the aqueous and organic fractions at the end of the series, a portion of the organic solvent is flashed overhead before a distillation step to recover furfural from the organic fraction. In an alternative approach, the organic fraction is catalytically decarbonylated to convert furfural to furan as described in the copending WO'783 application, and then the furan product and the solvent are separated by distillation.

### BRIEF DESCRIPTION OF THE DRAWINGS

FIG. 1 is a schematic representation of a “whole biomass” process according to the first aspect, wherein a fermentation of a mixed C₅/C₆ sugars feed from the hydrolysis of a whole biomass precedes an acid-catalyzed dehydration with a water-soluble acid in the presence of a low-boiling, substantially water-immiscible organic solvent to convert pentoses in the mixed C₅/C₆ sugars feed to furfural.

FIG. 2 is a schematic representation of one embodiment of a process for accomplishing the acid-catalyzed dehydration.

### DETAILED DESCRIPTION OF ILLUSTRATIVE EMBODIMENTS

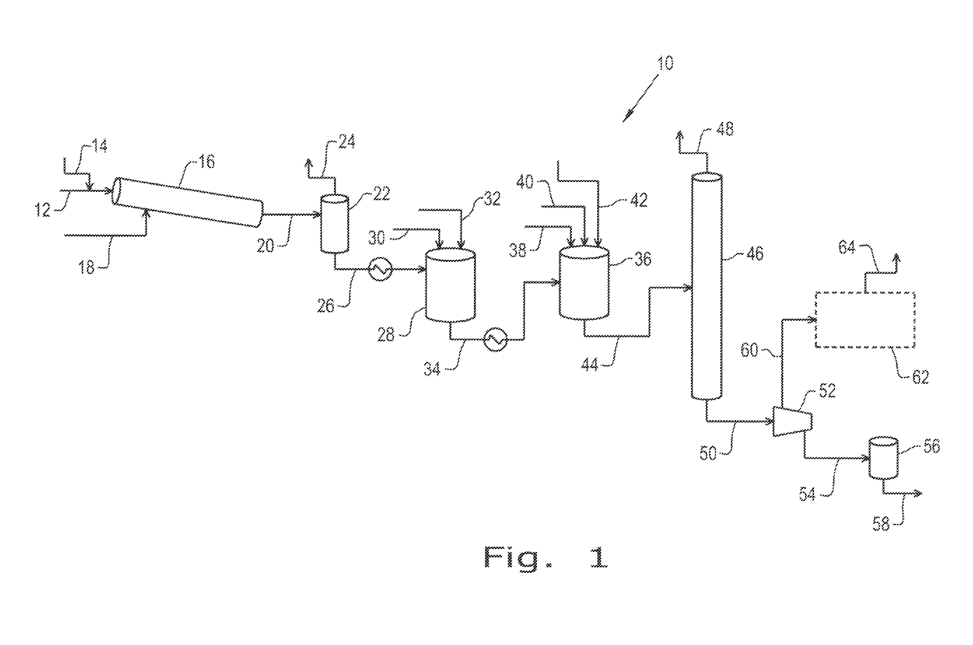

Turning now to FIG. 1, a process of the present invention is schematically illustrated according to a first aspect wherein hexoses in a mixed C₅/C₆ sugars feed are fermented to ethanol while pentoses in the mixed C₅/C₆ sugars feed undergo an acid-catalyzed dehydration and cyclization to produce furfural.

As an overview of the “whole biomass” embodiment 10 shown in FIG. 1, a lignocellulosic biomass 12 is combined with a water-soluble acid 14 in a digester 16, with steam 18 being added to provide heat for the digestion of the biomass 12. Corn kernel fiber is a readily available biomass in current corn-to-ethanol wet mills, so provides a convenient lignocellulosic biomass 12. Preferred water-soluble acids 14 for a whole biomass process will be those that have been historically used for producing furfural from corncobs and the like, for example, soluble inorganic acids such as sulfuric, phosphoric and hydrochloric acid; in alternate embodiments described below working with a mixed C₅/C₆ sugars feed from a prior fractionation method already involving an acid hydrolysis step, it will be appreciated that the preferred water-soluble acids may be or may include those already present in the aqueous sugars solution from the preceding fractionation method and, further, that the acids present may be sufficient for accomplishing the production of furfural from the pentoses and in a further optional embodiment of levulinic acid from the hexoses. Thus, for example, the water-soluble acids may be or may include C₁-C₂ acids selected from the group consisting of acetic acid, formic acid and mixtures of the same. Other acid catalysts, for example, AlCl₃ hexahydrate with hydrochloric acid, may also be used. Of course, it will be understood that additional acid may be supplied as well for the formation of furfural from the pentoses and further for the production of levulinic acid from the hexoses.

The biomass hydrolyzate 20 from digester 16 (or a hot mixed C₅/C₆ sugars feed from a prior fractionation method) then proceeds to a flash vessel 22 wherein excess water is flashed overhead in stream 24. A dewatered hydrolyzate stream 26 is then cooled to remove excess heat used for the digestion of the biomass 12 in the digester 16 as the dewatered hydrolyzate stream 26 enters the mashing, saccharification and fermentation section of the process 10.

The stream 26 then enters a vessel 28, wherein after partial neutralization with an added base 30 (for example, ammonium hydroxide) and addition of cellulase enzymes 32 (for example, α-amylase enzyme), a liquefied mash 34 comprising a mixture of pentose and hexose sugars and oligomers as well as some non-fermentable solids is produced. The liquefied mash 34 then proceeds to a fermentation vessel 36, wherein the mash 34 is combined according to conventional ethanol fermentation methods with a fermentative, ethanol-producing microorganism 38 (for example, a yeast such as saccharomyces cerevisiae, a bacteria or fungus) and additional enzymes 40 (for example, glucoamylase) to produce ethanol from the mash 34. The fermentation is controlled so as to obtain minimal conversion of the pentoses in the mash 34 to sugar alcohols, so that these pentoses can be subsequently converted to furfural; in the context of a conventional saccharomyces cerevisiae fermentation, for instance, we have found that this objective can be obtained by tracking the rate of carbon dioxide production over time, and stopping the fermentation as the carbon dioxide production rate approaches zero and as the more-readily fermented hexose sugars in the mash 34 have been depleted. It is expected that it should be possible in this regard to convert at least about 99.5 percent and preferably as high as about 99.9 percent of the hexoses in the initial sugars mixture without unduly risking loss of pentoses that would otherwise be available to convert to furfural and the products that can be made from furfural. Preferably, in any event, the fermentation is controlled so that at least about 90 percent of the pentoses remain unconverted, while more preferably at least about 95 percent of the pentoses remain unconverted, still more preferably at least about 99 percent of the pentoses remain unconverted and most preferably at least about 99.5 percent of the pentoses remain unconverted.

Where appropriate and desired to realize a more economic recovery of the ethanol thus produced in a subsequent distillation by reducing the reboiler duty therein, the hexose sugars in the mash 34 can be supplemented—with any requisite adjustment in the amounts and types of enzymes added in stream 40 and other appropriate adjustments in the fermentation conditions in vessel 36—by addition of liquefied starch via stream 42. In other embodiments, the ethanol produced in vessel 36 is sufficient in itself or when combined with ethanol from other fermenters (whether in parallel processes 10 or from other operations) to be economically distilled in the absence of the ethanol that would be produced from the added liquefied starch to omit the addition of starch to fermenter 36 via stream 42.

In still other embodiments, as mentioned in the summary above, the ethanol produced in vessel 36 will not be recovered through distillation as a commercial grade product but will be used to modify the properties of a low boiling, substantially water-immiscible organic solvent used in certain embodiments for the acid-catalyzed dehydration step in the subsequent production of furfural, and to improve the recovery of valuable hexose dehydration products in addition to furfural, namely, 5-hydroxymethylfurfural (HMF) and levulinic acid.

Where recovery of a commercial grade ethanol product, however, is desired, then a product 44 from the fermenter 36 including substantial ethanol, pentoses for the subsequent production of furfural, and certain non-fermentable solids is passed to a distillation column 46. The distillation of the product 44 therein provides a commercial grade ethanol product overhead as stream 48, typically and preferably being about 95 percent ethanol, while bottoms 50 comprising both the solubilized pentoses designated for producing furfural and non-fermentable solids passes to a solids-liquid separation, for example, a centrifugal separator 52. The solids 54 from separator 52 may be dried in a drier 56 to provide a high protein animal feed product 58, while solubilized pentoses from bottoms 50 are recovered from separator 52 in a liquid feed 60 to a subsequent production step 62 for producing a furfural product 64.

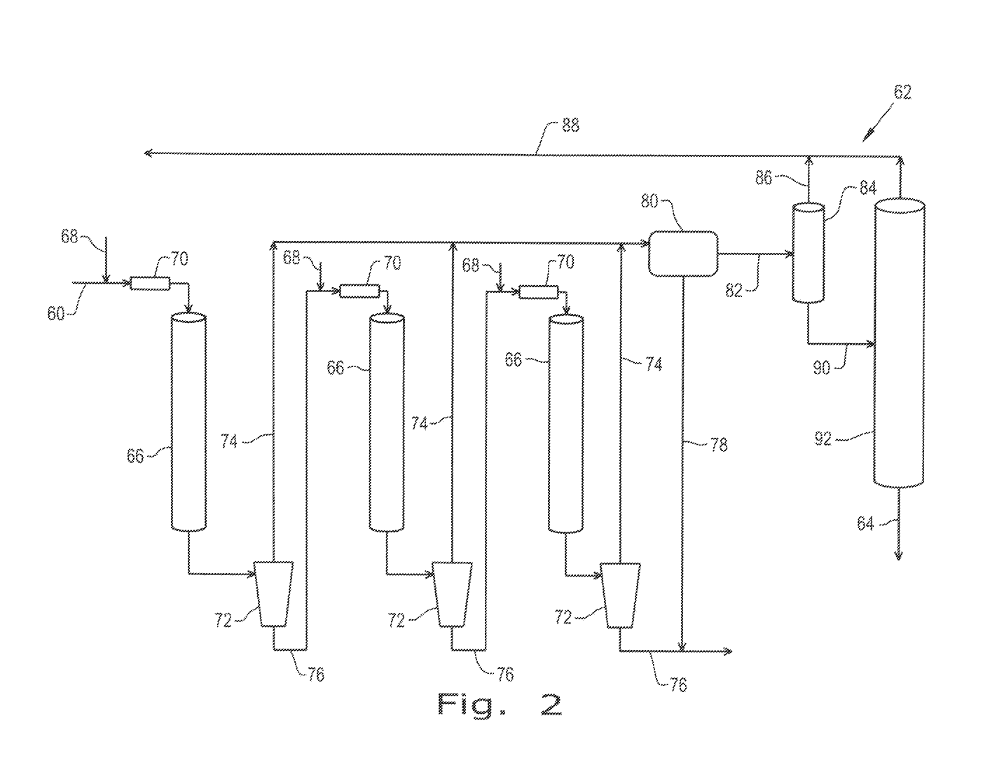

Turning now to FIG. 2, a schematic representation of one such process 62 is provided, wherein the liquid feed 60 including solubilized pentoses is acid-dehydrated in a plurality of reactors in series with an addition of a low-boiling, substantially water-immiscible organic solvent upstream of each reactor in the series. In the particular embodiment shown in FIG. 2, the acid dehydration of the pentoses to furfural is continuously accomplished in three reactor stages 66 in series. A low-boiling, substantially water-immiscible organic solvent is added in three corresponding increments 68 upstream of the reactor stages 66, with inline static mixers 70 being used to thoroughly mix the solvent and the liquid feed 60 upstream of the first reactor stage 66 and to thoroughly mix the solvent and the partially converted liquid feed after the first and second reactor stages 66. Preferred low-boiling, substantially water-immiscible solvents include toluene, ethanol, tetrahydrofuran and methyl tetrahydrofuran; the toluene and tetrahydrofuran provide obvious integration options when considered in relation to the use of furfural to make furan and subsequently THF from furan, while ethanol as described herein may be produced from hexoses in a mixed C₅/C₆ sugars feed from a prior biomass fractionation or from hydrolysis of a whole biomass (e.g., corn kernel fiber) and thus provides additional obvious integration options and benefits.

Interstage separators 72 between reactor stages 66 each function to recover an organic phase portion 74 comprising furfural formed by dehydration in the presence of the soluble acid catalyst added via stream 14 in the low-boiling, substantially water-immiscible organic solvent (and/or previously in a biomass fractionation process including acid hydrolysis), while aqueous phase portions 76 comprised of any unconverted five- and six-carbon sugars, salts, the water-soluble acid catalyst, 5-hydroxymethylfurfural and levulinic acid continue to a subsequent stage 66 or may be recycled for combination with liquid feed 60 at the start of the series of reactor stages 66 for further dehydration to a levulinic acid product. The collected organic phase portions 74 from the several interstage separators 72 are separated from any residual aqueous phase materials 78 in a decanter 80, with the decanted furfural/solvent mixture 82 proceeding to a flash vessel 84 to flash off a portion 86 of the solvent for recycle in solvent recycle stream 88. The remainder 90 is distilled in a distillation column 92 to remove the low-boiling, substantially water-immiscible solvent for recycle in the solvent recycle stream 88 and the furfural product stream 64.

In an alternate embodiment (not shown), the decanted furfural/solvent mixture 82 is catalytically decarbonylated to convert furfural to furan as described in our WO'783 application. In this application, decarbonylations were run on both synthetic 5% furfural in toluene feeds and on toluene extracts of the acid-catalyzed dehydration/cyclization furfural product of a pentose-containing fraction from biomass, using a 1% Pd/Al₂O₃ catalyst and a 2% Pd/C catalyst at a temperature ranging from 200 to 250 degrees Celsius for the synthetic feed examples and using the same palladium on alumina catalyst at 250 degrees Celsius for the actual dehydration feeds. Other catalysts could also be used, including supported and promoted or unpromoted platinum, rhodium, palladium and nickel catalysts. The furan decarbonylation product and the solvent are then separated by simple distillation. In this regard, whereas furfural has a boiling point of 161.7 degrees Celsius (compared to a boiling point of toluene at 110.6 deg. C), furan has a much lower boiling point of 31.3 degrees Celsius and so can be separated from toluene with considerably less energy being required.

The furan thus prepared and recovered can then be hydrogenated to produce tetrahydrofuran (THF), an important solvent and intermediate in the production of Spandex® elastomeric polyurethane fibers and other polymers. A number of catalysts and processes are known for this purpose. For example, U.S. Pat. No. 2,846,449 to Banford et al. prescribe finely divided nickel, platinum or palladium in the pure state or on an inert support, with foraminous or Raney® nickel sponge metal catalyst, and finely divided reduced nickel or kieselguhr being listed as preferred catalyst choices.

Conventionally, THF has been made from non-renewable resources, though a significant amount of research has been carried out over a number of years in relation to dehydrating pentoses found in or obtained from biomass to furfural, decarbonylating the furfural to furan, and then finally hydrogenating the furan to THF.

As evidenced, however, by a series of related filings by one of the largest producers and developers of THF technology, see, for example, US 2013/0168227, US 2013/0172581, US 2013/0172582, US 2013/0172583, US 2013/0172584, US 2013/0172585, US2013/0109869, US 2012/0157697 and US 2011/0213112, there remains a substantial need for further improvement in methods for producing furfural from biomass that will be conducive to the economical realization of a furan product that can be hydrogenated to THF.

US 2013/0172584 to Corbin et al. is representative of the approach taken in these filings, wherein furfural is produced by mixing an aqueous feedstock solution containing C₅ sugars and/or C₆ sugars with a heated high boiling water-miscible solvent, such as sulfolane, and a solid acid catalyst. More particularly, a reactive distillation process is described wherein the aqueous feedstock solution is added to a reaction vessel containing the solid acid catalyst in a high boiling water miscible organic solvent, and the dehydration reaction is conducted in the vessel at a temperature of from 100 to 250 degrees Celsius and a pressure from 0 MPa to 0.21 MPa. A mixture of water and furfural is removed overhead from the distillation column located on top of the reaction vessel, via reflux through a multistage distillation to “minimize” loss of the water-miscible organic solvent overhead, while the high boiling water-miscible organic solvent is used to keep byproducts such as humins dissolved and to prevent their deposition on the solid catalysts. In a continuous embodiment described in the reference, at least a portion of the contents of the reaction vessel are pumped through a filter or screen to prevent aspiration of the solid acid catalyst, and then diluted with either aqueous feedstock solution water or simply water to precipitate the water-insoluble byproducts from solution in the high-boiling water-miscible solvent. These water-insoluble byproducts are then removed by filtration or centrifugation.

Recovery of the high-boiling water-miscible solvent by distillation would, however, be of considerable expense, while recycling the solvent would likely involve the buildup of byproducts in the system and perhaps of residual humins not removed by precipitation and filtration or centrifugation. Where toluene is preferably used as the low-boiling, substantially water-immiscible solvent for the present invention, a number of other benefits may be realized as well. Firstly, toluene is a much less expensive solvent than a high-boiling water-miscible solvent such as sulfolane. The toluene conveniently can be used in a subsequent process according to our WO'783 application or may be readily separated from the furfural, while in comparison some loss of the high boiling water-miscible solvent in the distillation of the furfural/water overhead is apparently to be accepted in Corbin et al's process and while further costly high boiling solvent will likely be lost in the further steps to remove humins, the solid catalyst and salts from the bottoms. Further, the processing of the bottoms to remove humins, the solid catalyst and salts as well as the presumed regeneration of the solid acid catalyst represent substantial additional processing costs, whereas the use of a low-boiling, water-immiscible solvent permits the salts and acid catalyst to be recycled directly for further use. Finally, residual hexoses in Corbin et al's process can form additional humins, making the downstream processing and recovery of furfural more complex.

What is claimed is:

1. A process for making both ethanol and furfural from a mixture of pentoses and hexoses, comprising:
a) fermenting a mixture of hexoses and pentoses to convert hexoses in the mixture to ethanol;
b) concluding the fermentation prior to any substantial conversion of pentoses in the mixture to sugar alcohols;
c) separating unconverted pentoses in the mixture from ethanol formed in the fermentation, to yield an ethanol product; and
d) dehydrating and cyclizing the separated unconverted pentoses to furfural; and
e) recovering a furfural product.

2. A process according to claim 1, further comprising combining ethanol from a starch fermentation with ethanol from the fermentation of hexoses in the mixture of pentoses and hexoses, prior to the separation step c).

3. A process according to claim 2, wherein starch is combined with hexoses in the mixture of pentoses and hexoses, and the starch and hexoses are fermented together to provide the ethanol product.

4. A process according to claim 2, wherein the dehydration and cyclization of pentoses to furfural is accomplished using a water-soluble acid catalyst at elevated temperatures in the presence of a low-boiling, substantially water-immiscible organic solvent.

5. A process according to claim 1, wherein the dehydration and cyclization of pentoses to furfural is accomplished using a water-soluble acid catalyst at elevated temperatures in the presence of a low-boiling, substantially water-immiscible organic solvent.

6. A process according to claim 4, wherein the low-boiling, substantially water-immiscible organic solvent is selected from toluene, ethanol, tetrahydrofuran and methyl tetrahydrofuran.

7. A process according to claim 5, wherein the low-boiling, substantially water-immiscible organic solvent is selected from toluene, ethanol, tetrahydrofuran and methyl tetrahydrofuran.

8. A process according to claim 4, wherein the water-soluble acid catalyst is selected from sulfuric acid, phosphoric acid, hydrochloric acids, acetic acid, formic acid, AlCl₃.6H₂O and mixtures of any of these.

9. A process according to claim 5, wherein the water-soluble acid catalyst is selected from sulfuric acid, phosphoric acid, hydrochloric acids, acetic acid, formic acid, AlCl₃.6H₂O and mixtures of any of these.

10. A process according to claim 4, wherein furfural formed in the process is extracted into the low-boiling, water-immiscible solvent.

11. A process according to claim 5, wherein furfural formed in the process is extracted into the low-boiling, water-immiscible solvent.

12. A process according to claim 10, wherein the dehydration and cyclization of pentoses to furfural and extraction of furfural into the low-boiling, water-immiscible solvent is accomplished in a series of reactors with addition of low-boiling, water-immiscible solvent upstream of each reactor and with recovery in part of the furfural in an organic phase portion following each reactor.

13. A process according to claim 11, wherein the dehydration and cyclization of pentoses to furfural and extraction of furfural into the low-boiling, water-immiscible solvent is accomplished in a series of reactors with addition of low-boiling, water-immiscible solvent upstream of each reactor and with recovery in part of the furfural in an organic phase portion following each reactor.

14. A process according to claim 12, further comprising collecting the organic phase portions, flashing off low-boiling, water-immiscible solvent from the collected organic phase portions and then distilling the remainder to provide a furfural product.

15. A process according to claim 13, further comprising collecting the organic phase portions, flashing off low-boiling, water-immiscible solvent from the collected organic phase portions and then distilling the remainder to provide a furfural product.

16. A process according to claim 12, further comprising collecting aqueous phase portions including the water-soluble acid catalyst following each reactor and recycling the collected aqueous phase portions for reuse in the dehydration and cyclization step.

17. A process according to claim 13, further comprising collecting aqueous phase portions including the water-soluble acid catalyst following each reactor and recycling the collected aqueous phase portions for reuse in the dehydration and cyclization step.

18. A process according to claim 12, wherein the low-boiling, water-immiscible solvent is toluene and further comprising catalytically decarbonylating furfural extracted into the toluene to provide furan.

19. A process according to claim 13, wherein the low-boiling, water-immiscible solvent is toluene and further comprising catalytically decarbonylating furfural extracted into the toluene to provide furan.

20. A process according to claim 18, further comprising catalytically hydrogenating furan to tetrahydrofuran.

21. A process according to claim 19, further comprising catalytically hydrogenating furan to tetrahydrofuran.

22. A process according to claim 1, wherein the mixture of pentoses and hexoses is obtained from a fractionation of a lignocellulosic biomass including hydrolysis of celluloses and hemicelluloses in the biomass.

23. A process according to claim 22, wherein the hydrolysis of celluloses and hemicelluloses in the biomass is accomplished by a water-soluble acid that is subsequently used for catalyzing the dehydration and cyclization of pentoses to furfural.

24. A process according to claim 1, wherein the mixture of pentoses and hexoses is the material produced by the acid hydrolysis at an elevated temperature of a whole biomass.

25. A process according to claim 25, wherein the acid used for the acid hydrolysis of the whole biomass is a water-soluble acid that is subsequently used for catalyzing the dehydration and cyclization of pentoses to furfural.

26. A process for making furfural from a mixture of pentoses and hexoses, comprising:
a) fermenting a mixture of hexoses and pentoses to convert hexoses in the mixture to ethanol;
b) concluding the fermentation prior to any substantial conversion of pentoses in the mixture to sugar alcohols;
c) dehydrating and cyclizing unconverted pentoses in the fermentation broth to furfural; and
d) separating out furfural so formed.

27. A process according to claim 26, wherein the dehydration and cyclization of pentoses to furfural is accomplished using a water-soluble acid catalyst at elevated temperatures in the presence of a low-boiling, substantially water-immiscible organic solvent added to the fermentation broth, into which furfural is extracted.

28. A process according to claim 27, wherein the formation and extraction of furfural are done continuously and concurrently.

29. A process according to claim 28, wherein the dehydration and cyclization of pentoses to furfural and extraction of furfural into the low-boiling, water-immiscible solvent are continuously accomplished in a series of reactors in sequence with an addition of the low-boiling, water-immiscible solvent upstream of each reactor and with recovery in part of the furfural in an organic phase portion following each reactor.

30. A process according to claim 29, further comprising collecting the organic phase portions, flashing off low-boiling, water-immiscible solvent from the collected organic phase portions and then distilling the remainder to provide a furfural product.

31. A process according to claim 29, wherein the low-boiling, water-immiscible solvent is toluene and further comprising catalytically decarbonylating furfural extracted into the toluene to provide furan.

32. A process according to claim 31, further comprising catalytically hydrogenating furan to tetrahydrofuran.

33. A process according to claim 26, wherein the mixture of pentoses and hexoses is obtained from a fractionation of a lignocellulosic biomass including hydrolysis of celluloses and hemicelluloses in the biomass.

34. A process according to claim 33, wherein the hydrolysis of celluloses and hemicelluloses in the biomass is accomplished by a water-soluble acid that is subsequently used for catalyzing the dehydration and cyclization of pentoses to furfural.

35. A process according to claim 26, wherein the mixture of pentoses and hexoses is the material produced by the acid hydrolysis at an elevated temperature of a whole biomass.

36. A process according to claim 35, wherein the whole biomass comprises corn kernel fiber.

37. A process according to claim 35, wherein the acid used for the acid hydrolysis of the whole biomass is a water-soluble acid that is subsequently used for catalyzing the dehydration and cyclization of pentoses to furfural.

38. A continuous process for making furfural from a mixture of pentoses and hexoses, comprising a) dehydrating and cyclizing pentoses in the mixture to furfural with a water-soluble acid catalyst in a series of reactors in sequence, b) adding a portion of a low-boiling, water-immiscible solvent upstream of each reactor for extracting furfural selectively into an organic phase portion following each reactor, c) collecting the organic phase portions and d) flashing off low-boiling, water-immiscible solvent from the collected organic phase portions and then e) distilling the remainder to provide a furfural product.

39. A continuous process for making furan from a mixture of pentoses and hexoses, comprising a) dehydrating and cyclizing pentoses in the mixture to furfural with a water-soluble acid catalyst in a series of reactors in sequence, b) adding a portion of a low-boiling, water-immiscible solvent upstream of each reactor for extracting furfural selectively into an organic phase portion following each reactor, c) collecting the organic phase portions, d) catalytically decarbonylating furfural in the collected organic phase portions to furan, and e) separating, by distillation, a furan product from a remainder including the low-boiling, water-immiscible solvent.

40. A process according to claim 38, further comprising hydrogenating the furan product to tetrahydrofuran.
